SEQUENCE LISTING (1 sequence)

SEQ ID NO: 1 - DNA, 1513 bp, Artificial Sequence
  Synthetic
aaaaccgaattttgctgggtgggctaacgatatccgcctgatgcgtgaacgtgacggacgtaacgaagacaaccacgcattgctgttctgagctaacaccgtgcgtgttgacaattttacctctggcggtgataatggttgcagcaagcaataattttgtttaactttaagaaggagatatacaatgaaaatcgaagaaggtaaactggtaatctggattaacggcgataaaggctataacggcctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactcgtatcaccaagggtggatctggatgttgtcctggctgttgctgatgaataactgaataggggatcccgactggcgagagccaggtaacgaatggatccccgagctcgagcaaagcccgccgaaaggcgggcttttctgtccttgagagtcgggcattgtcttcgctccttccggtgggcgcggggcatgactatcgtcgccgcacttatgactgtgttctttatcat

(19) United States
(12) Patent Application Publication
(10) Pub. No.: US 2023/0193337 A1
(43) Pub. Date: Jun. 22, 2023

(54) Anaerobic Cell-Free Systems and Environments and Methods for Making and Using Same

(71) Applicant: Synvitrobio, Inc., San Francisco, CA (US)

(72) Inventors: Zachary Z. Sun, San Francisco, CA (US); Dan E. Robertson, San Francisco, CA (US); Kelly S. Trego, San Francisco, CA (US); Abel C. Chiao, IV, San Francisco, CA (US); Louis E. Metzger, IV, San Francisco, CA (US)

(21) Appl. No.: 16/757,181

(22) PCT Filed: Oct. 19, 2018
(86) PCT No.: PCT/US18/56672

§ 371 Date: Apr. 17, 2020

Related U.S. Application Data
(60) Provisional application No. 62/574,570, filed on Oct. 19, 2017.
(60) Provisional application No. 62/576,157, filed on Oct. 24, 2017.

(51) Int. Cl. C12P 21/00; C12N 9/08; C12N 9/04

(52) U.S. Cl. CPC C12P 21/00; C12N 9/0065; C12Y 111/01006; C12N 9/0006; C12Y 101/0301

(57) ABSTRACT

The present disclosure relates to cell-free compositions and methods for making and using the same. In one aspect, the composition includes: an extract derived from one or more organisms; one or more proteins of interest, wherein the one or more proteins are expressed from one or more nucleic acids exogenous to the extract and/or by the one or more organisms, wherein preferably the one or more proteins react with a substrate to produce a product; and one or more O2, O—, or H₂O₂ scavengers. The composition may be oxygen-deprived. The composition may also include an energy recycling system.

### CROSS-REFERENCE TO RELATED APPLICATIONS

This application claims priority to and the benefit of U.S. Provisional Application Nos. 62/574,570 filed Oct. 19, 2017 and 62/576,157 filed Oct. 24, 2017, both of which are incorporated by reference herein in their entireties.

### GOVERNMENT LICENSE RIGHTS

This invention was made with government support under W911NF-17P-0059 awarded by the United States Army Research Office. The government has certain rights in the invention.

### FIELD

The disclosure relates to cell-free compositions and use thereof, especially in the production of proteins and molecules in an anaerobic environment.

### BACKGROUND

Many proteins with industrial and therapeutic use are isolated from organisms in oxygen poor environments. Organisms can include those that live in deep sea vents, oil and gas wells, high salinity environments, and anaerobic environments. Despite efforts and progress, current approaches to perform isolation are often laborious, costly and difficult.

Cell-free systems provide a prototyping environment to express these proteins functionally and further engineer them into downstream systems. The key idea is that the cell lysate acts as a working chemical factory. Its components are catalysts that are activated when provided with essential substrates (e.g., amino acids, nucleotides energy substrates, cofactors, and salts). Upon incubation, these mini-factories take user-supplied recombinant DNA to synthesize and fold desired proteins, which can then execute behaviors or interesting metabolic functions. An in vitro transcription-translation cell-free system has been developed which allows for the rapid prototyping of genetic constructs in an environment that behaves similarly to a cell. One of the main purposes of working in vitro is to be able to generate fast speeds—in vitro, reactions can take 8 hours and can scale to thousands of reactions a day, a multi-told improvement over similar reactions in cells (Sun et al. 2014).

Currently protein production from anaerobes occurs through either isolation from the organism or through heterologous expression. The former requires knowledge of growth conditions for the anaerobe, many of which are un-cultivable. The latter requires the protein to express in a non-native host, for which cofactors/chaperones or conditions (e.g., temperature, pH) may not be present and/or correct for functional expression. Producing multiple proteins, which also require understanding native regulatory elements (e.g., promoters, repressors, activators), further increases challenge. There are applications where anaerobic conditions are required to produce proteins, enzymes, and molecules of interest, or to run pathways or genetic circuits. Anaerobic conditions can be achieved using physical methods (e.g., culturing in anaerobic conditions, running reactions in anaerobic hoods), although these conditions may interfere with cell-free system efficiency or may be infeasible or non-economical to conduct at scale.

Thus, a need exists for improved cell-free systems that can achieve anaerobic conditions, such as the cell-free systems disclosed herein.

### SUMMARY

Disclosed herein are improved in vitro anaerobic cell-free systems and environments and use thereof.

In a first aspect, a composition for in vitro transcription and translation is provided. The composition includes: an extract derived from one or more organisms; a template nucleic acid comprising a gene or gene portion of interest; and one or more O₂, O⁻, or H₂O₂ scavengers. The composition may be oxygen-deprived or anaerobic. The one or more organisms may be selected from the group consisting of: bacteria, archaea, plants, and animals. The one or more organisms may be selected from the group consisting of: extremophiles and Clostridium.

The one or more O₂, O⁻, or H₂O₂ scavengers may bind O₂, O⁻, or H₂O₂. The one or more O₂, O⁻, or H₂O₂ scavengers may biochemically convert O₂, O⁻, or H₂O₂ into another molecule. The one or more O₂, O⁻, or H₂O₂ scavengers maybe selected from the group consisting of: catalase, superoxide dismutase, peroxidase, hemoglobin, myoglobin, porphyrin, oxidase, oxygenase, rubisco, and homologs or variants thereof. The scavengers may include a transition metal. The transition metal may be in a heme group. The scavengers may be selected from the group consisting of: glucose, glucose oxidase, pyranose oxidase, and catalase. The scavengers may De selected from the group consisting of: protocatechuate 3,4-dioxygenase and protocatechuic acid.

The composition may further include an energy recycling system. The energy recycling system may include one or more components selected from the group consisting of: components for providing redox potential, components for providing phosphate potential, and combinations thereof.

The composition may further include one or more additives, wherein the one or more additives include one or more cofactors, enzymes, and other reagents necessary for transcription and/or translation.

In the composition, the template nucleic acid may include a gene derived from an extremophile or anaerobe.

In the composition, the extract may include one or more of the following: a whole cell extract, a nuclear extract, a cytoplasmic extract, and mixtures thereof. The extract may include a cell lysate.

A second aspect relates to a method for in vitro protein synthesis in a transcription and translation system. The method includes: (a) preparing a reaction mixture including: (i) an extract derived from one or more organisms; (ii) a template nucleic acid comprising a gene or gene portion of interest; and (iii) one or more O₂, O⁻, or H₂O₂ scavengers; and (b) expressing and isolating the protein from the reaction mixture.

The reaction mixture in the method may be oxygen-deprived or anaerobic. The one or more O₂, O⁻, or H₂O₂ scavengers may bind O₂, O⁻, or H₂O₂, or may biochemically convert O₂, O⁻, or H₂O₂ into a different molecule. The one or more O₂, O⁻, or H₂O₂ scavengers may be selected from the group consisting of: catalase, superoxide dismutase, peroxidase, hemoglobin, myoglobin, porphyrin, oxidase, oxygenase, rubisco, and homologs or variants thereof. The reaction mixture in the method may further include an energy recycling system.

A further aspect relates to a composition for in vitro transcription and translation, including: a. a first set of cofactors, enzymes, and other reagents necessary for transcription and/or translation; b. a second set of cofactors, enzymes, and other reagents necessary for energy recycling; c. a template nucleic acid comprising a gene or gene portion of interest; and d. one or more O₂, O⁻, or H₂O₂ scavengers. In the composition, the first set of proteins, enzymes, and other reagents necessary for transcription and/or translation may be wholly or partially provided by an extract derived from one or more organisms. The first set or proteins, enzymes, and other reagents necessary for transcription and/or translation may be wholly or partially provided from a fully or partially purified source. The second set of proteins, enzymes, and other reagents necessary for energy recycling may be wholly or partially provided by an extract derived from one or more organisms. The second set of proteins, enzymes, and other reagents necessary for energy recycling may be wholly or partially provided from a fully or partially purified source.

### BRIEF DESCRIPTION OF THE DRAWINGS

FIG. 1 provides an overview of cell-free expression. In cell-free expression, a host is converted into a lysate and supplied with factors to enable the conversion of DNA to mRNA and protein.

FIG. 2 provides a comparison of traditional heterologous expression to cell-free expression.

FIG. 3 demonstrates a sample oxygen scavenging system, composed of catalase, glucose oxidase, and glucose.

FIG. 4 shows the visual difference between a E. coli based cell-free expression reaction supplemented with oxygen scavenging (left) compared to one without oxygen scavenging (right). The color change, showing the oxidation state of free iron in solution, demonstrates the successful reduction of oxygen in the cell-free expression reaction. The color change is indicated by the units of intensity (higher is brighter), as measured by ImageJ software at the point at the black arrow.

FIG. 5 shows 8 conditions for a E. coli based cell-free expression reaction supplemented with or without oxygen scavenging (OS). Different DNA's (either expressing fluorescent GFP or a non-fluorescent coding sequence (CDS)) are run overnight at 200 μL at 29° C. Different energy regeneration systems are used: either CP/CK, which is oxygen-independent, or glutamate, which is oxygen-dependent. The resulting reaction is visualized (top), where color change, showing the oxidation state of free iron in solution, demonstrates the successful reduction of oxygen in the cell-free expression reaction. Black solid arrows, units of intensity (u.), higher is brighter, as measured by ImageJ at the point of the black arrow. Bot., bottom of tube. Dotted black arrow, unit of intensity at interface between bottom liquid and airspace. 10 μL of each reaction is then visualized on a Biotek Synergy 2 plate reader at 485/528 ex/em for signal correlated to a GFP standard.

FIG. 6 shows active expression and detection of Mannose Binding Protein (MBP) in a oxygen-independent method for cell-free expression reactions. DNA expressing MBP or no DNA control is expressed overnight at 200 μL at 29° C. in a 0.2 mL tube in the presence of 1% FluoroTect™, gamS, oxygen scavenging solution, and other additions. Shown is a 4-12% MES SDS-PAGE gel of 2 μL of each sample, imaged for FluoroTect™ intensity, with the band size for MBP indicated.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Disclosed herein are compositions and formulations of anaerobic cell-free transcription and translation systems (also termed anaerobic in vitro transcription and translation systems), and methods of using. In an embodiment, anaerobic cell-free transcription and translation systems described herein include: an extract derived from one or more organisms; a template nucleic acid; and one or more O₂, O⁻, or H₂O₂ scavengers (also termed scavengers herein). They may also include an energy recycling system for providing phosphate potential or redox potential. In an embodiment, anaerobic cell-free transcription and translation systems described herein include: a first set of cofactors, enzymes, and other reagents necessary for transcription and/or translation; a second set of cofactors, enzymes, and other reagents necessary for energy recycling; a template nucleic acid comprising a gene or gene portion of interest; and one or more O₂, O⁻, or H₂O₂ scavengers.

Such anaerobic cell-free systems may emulate the anaerobic environments of archaeal, bacterial or eukaryotic sources operating in biomes at environmental extremes, e.g., temperature, pH, salinity, redox potential, etc., or from organisms or communities evolved in niches that selected for trophic variations of central and peripheral metabolism, e.g., auto-, chemo-, hetero-, phototrophic, etc.

Compartments, mechanisms and modes of existence have evolved to drive and protect life systems under extensive, e.g., temperature, pH, and intensive conditions, e.g., the niche chemistry of the extant environment, and to utilize available nutrients, redox couples, etc. many of these mechanisms dominated life systems in the pre-oxygen atmosphere (>3B years) or evolved to protect these mechanisms in the expanding oxygen atmosphere.

An estimated 70 components participate in the mechanism of transcription and translation in E. coli. Exergonic metabolic pathways supply energy currency to drive the endergonic reactions of gene expression in the form of phosphate potential ([ATP]/[ADP]+[Pi]), and redox potential ([NAD(P)H]/[NAD(P+)]). The number, identities, specificities and mechanisms of expression system components may vary across phyla and reflect evolution under environmentally select conditions.

Likewise, genes that evolved in anaerobic or extremophile organisms may have cryptic sequence elements that regulate their expression, or may code for proteins that are sensitive to ex vivo conditions. Thus, without wishing to be bound by theory, it has been hypothesized that the fidelity and yield of in vitro gene expression will be optimal within the context of a phylogenically similar TXTL system with genes engineered for optimum expression, or a TXTL system that emulates the source organism (eg. anaerobic).

The improved in vitro transcription/translation (TXTL) system disclosed herein can efficiently catalyze information flow from DNA to cellular function. It improves upon prior systems by broadening its utility for bioengineering and biodiscovery. In some embodiments, the systems and compositions disclosed herein are designed to promote the expression of genetic material from anaerobic or extremophile organisms by encouraging an anaerobic environment. However, the compositional modifications can be implemented for an in vitro system derived from any organism. When used for biodiscovery, the compositions and methods disclosed herein can remove largely unsolved barriers to conventional gene expression in heterologous hosts, opening vast areas of gene sequence space for exploration; via expression of genes from uncultured organisms, microbiomes, libraries of cryptic genes and clusters.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to reter to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer therebetween.

The term “additive” refers to an addition, whether chemical or biological in nature, whether natural or synthetic or, that is provided to a system. Examples include but are not limited to enzymes, oxidases, oxygenases, sugars, betaine, cyclodextrans, solvents, alcohols, proteins, enzymes, and nucleic acids.

As used herein, the terms “nucleic acid,” “nucleic acid molecule” and “polynucleotide” may be used interchangeably and include both single-stranded (ss) and double-stranded (ds) RNA, DNA and RNA:DNA hybrids. These terms are intended to include, but are not limited to, a polymeric form of nucleotides that may have various lengths, including deoxyribonucleotides and/or ribonucleotides, or analogs or modifications thereof. A nucleic acid molecule may encode a full-length polypeptide or RNA or a fragment of any length thereof, or may be non-coding.

Nucleic acids can be naturally-occurring or synthetic polymeric forms of nucleotides. The nucleic acid molecules of the present disclosure may be formed from naturally-occurring nucleotides, for example forming deoxyribonucleic acid (DNA) or ribonucleic acid (RNA) molecules. Alternatively, the naturally-occurring oligonucleotides may include structural modifications to alter their properties, such as in peptide nucleic acids (PNA) or in locked nucleic acids (LNA). The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. Modifications can also include phosphorothioated bases for increased stability.

As used herein, unless otherwise stated, the term “transcription” refers to the synthesis of RNA from a DNA template; the term “translation” refers to the synthesis of a polypeptide from an mRNA template. Translation in general is regulated by the sequence and structure of the 5′ untranslated region (5′-UTR) of the mRNA transcript. One regulatory sequence is the ribosome binding site (RBS), which promotes efficient and accurate translation of mRNA. The prokaryotic RBS is the Shine-Dalgarno sequence, a purine-rich sequence of 5′-UTR that is complementary to the UCCU core sequence of the 3′-end of 16S rRNA (located within the 30S small ribosomal subunit). Various Shine-Dalgarno sequences have been found in prokaryotic mRNAs and generally lie about 10 nucleotides upstream from the AUG start codon. Activity of a RBS can be influenced by the length and nucleotide composition of the spacer separating the RBS and the initiator AUG. In eukaryotes, the Kozak sequence lies within a short 5′ untranslated region and directs translation of mRNA. An mRNA lacking the Kozak consensus sequence may also be translated efficiently in an in vitro system if it possesses a moderately long 5′-UTR that lacks stable secondary structure. While E. coli ribosome preferentially recognizes the Shine-Dalgarno sequence, eukaryotic ribosomes (such as those found in retic lysate) can efficiently use either the Shine-Dalgarno or the Kozak ribosomal binding sites.

As used herein, the term “host” or “host cell” refers to any prokaryotic or eukaryotic single cell (e.g., yeast, bacterial, archaeal, etc.) cell or organism. The host cell can be a recipient of a replicable expression vector, cloning vector or any heterologous nucleic acid molecule. Host cells may be prokaryotic cells such as species of the genus Escherichia or Lactobacillus, or eukaryotic single cell organism such as yeast. The heterologous nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Green & Sambrook, 2012, Molecular Cloning: A laboratory manual, 4th ed., Cold Spring Harbor Laboratory Press, New York, which are hereby incorporated by reference herein in their entireties.

As used herein, the term “selectable marker” or “reporter” refers to a gene, operon, or protein that upon expression in a host cell or organism, can confer certain characteristics that can be relatively easily selected, identified and/or measured. Reporter genes are often used as an indication of whether a certain gene has been introduced into or expressed in the host cell or organism. Examples, without limitation, of commonly used reporters include: antibiotic resistance (“abR”) genes, fluorescent proteins, auxotropic selection modules, P-galactosidase (encoded by the bacterial gene lacZ), luciferase (from lightning bugs), chloramphenicol acetyltransferase (CAT; from bacteria), GUS (β-glucuronidase; commonly used in plants) green fluorescent protein (GFP; from jelly fish), and red fluorescent protein (RFP). Typically host cells expressing the selectable marker are protected from a selective agent that is toxic or inhibitory to cell growth.

The term “engineer,” “engineering” or “engineered,” as used herein, refers to genetic manipulation or modification of biomolecules such as DNA, RNA and/or protein, or like technique commonly known in the biotechnology art.

As described herein, “genetic module” and “genetic element” may be used interchangeably and refer to any coding and/or non-coding nucleic acid sequence. Genetic modules may be operons, genes, gene fragments, promoters, exons, introns, regulatory sequences, tags, or any combination thereof. In some embodiments, a genetic module refers to one or more of coding sequence, promoter, terminator, untranslated region, ribosome binding site, polyadenlylation tail, leader, signal sequence, vector and any combination of the foregoing. In certain embodiments, a genetic module can be a transcription unit as defined herein.

As used herein, a “homolog” of a gene or protein, “homology,” or “homologous” refers to its functional equivalent in another species. The terms “substantial identity” in the context of a peptide indicates that a peptide comprises a sequence with at least 70% sequence identity to a reference sequence, for example at least 80%, at least 85%, at least 90%, at least 95%, at least 96%, at least 97%, at least 98%, or at least 99% sequence identity to the reference sequence, over a specified comparison window. Optionally, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions that are not identical may differ by conservative amino acid changes.

As used herein, a “variant” of a gene or nucleic acid sequence is a sequence having at least 10% identity with the referenced gene or nucleic acid sequence, and can include one or more base deletions, additions, or substitutions with respect to the referenced sequence. The differences in the sequences may by the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” of the original sequence. A “variant” of a peptide or protein is a peptide or protein sequence that varies at one or more amino acid positions with respect to the reference peptide or protein. A variant can be a naturally-occurring variant or can be the result of spontaneous, induced, or genetically engineered mutation(s) to the nucleic acid molecule encoding the variant peptide or protein. A variant peptide can also be a chemically synthesized variant.

As used herein, “oxygen-deprived”, refers to an environment in which the oxygen has been substantially removed. By way of example only, the oxygen concentration may be less than 5 ppm under standard temperature and pressure (STP), or the oxygen concentration may be less than 2 ppm, or the oxygen concentration may be less than 1 ppm, or the oxygen concentration may be less than 0.01 ppm, or the oxygen concentration may be less than 0.001 ppm. An anaerobic environment is an example of an oxygen-deprived environment.

As used herein, “anaerobe” means any eukaryotic or prokaryotic cell whose growth can be inhibited by the presence of free oxygen, including but not limited to all cells traditionally classified as obligate anaerobes and microaerophiles, and those obligate aerobes and facultative anaerobes which are inhibited by pure oxygen.

As used herein, “extremophile” refers to an organism that exhibits optimal growth under extreme environment conditions. Extremophiles include acidophiles, alkaliphiles, halophiles, thermophiles (including hyerthermophiles, which are typically found in an environment that has a temperature of above 80° C.), metalotolerant organisms, osmophiles, and xerophiles.

As used herein, “oxygen, oxygen radicals, and/or hydrogen peroxide” and “O₂, O⁻, and/or H₂O₂” are used interchangeably herein.

Other terms used in the fields of recombinant nucleic acid technology and molecular and cell biology as used herein will be generally understood by one of ordinary skill in the applicable arts.

### Composition of In Vitro Transcription and Translation System

The in vitro transcription and translation system is a system that is able to conduct transcription and translation outside of the context of a cell. In some embodiments, this system is also referred to as “cell-free system”, “cell-free transcription and translation”, “TX-TL”, “TXTL”, “TX/TL”, “extract systems”, “in vitro system”, “ITT”, or “artificial cells.” Exemplary in vitro transcription and translation systems include purified or partially purified protein systems that are made from hosts, purified or partially purified protein systems that are not made from hosts, and protein systems made from a host strain that is formed as an “extract”. In an embodiment, extracts include whole-cell extracts, nuclear extracts, cytoplasmic extracts, combinations thereof, and the like. Whole-cell extracts are also termed lysates herein. Lysates, and lysate systems, described herein, are intended to be non-limiting examples of extracts; where lysate is described herein, it is contemplated that other extracts, or extracts and protein combinations, may be used.

In an embodiment, a cell-free system may include a combination of cytoplasmic and/or nuclear components from cells. The components may include extracts, purified components, or combinations thereof. The extracts, purified components, or combinations thereof include reactants for protein synthesis, transcription, translation, DNA replication and/or additional biological reactions occurring in a cellular environment identifiable by a person skilled in the art.

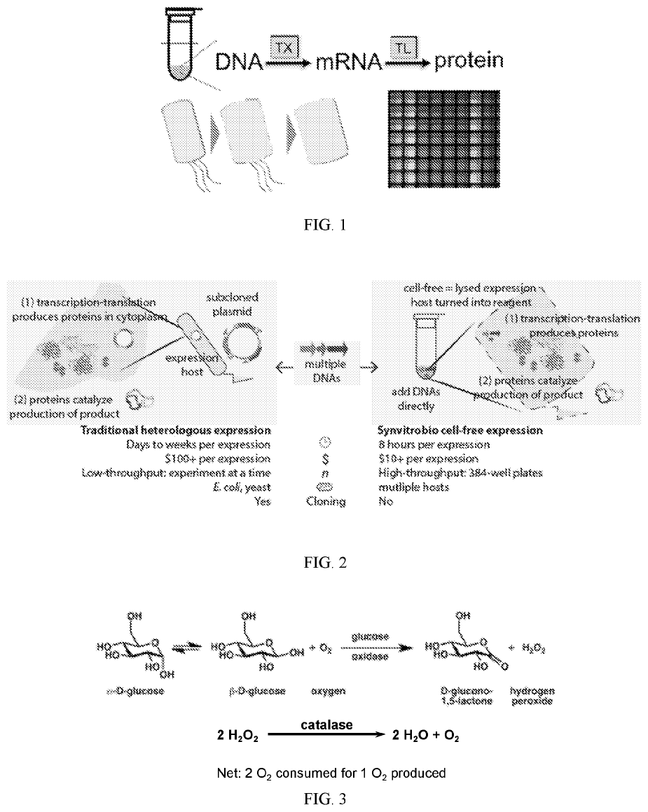

Cell-free transcription-translation is described in FIG. 1. Top, cell-free expression that takes in DNA and produces protein that catalyzes reactions. Bottom, diagram of cell-free production and representative data collected in 384-well plate format of GFP expression. Cell-free approaches contrasted to cellular approaches are described in FIG. 2. Cell-free platform allows for protein expression from multiple genes without live cells. Cell-free production biotechnology methods produce lysates from prokaryotic cells that are able to take recombinant DNA as input and conduct coupled transcription and translation to output enzymatically active protein. Cell-free systems take only 8 hours to express, rather than days to weeks in cells, since there is no need for cloning and transformation. They are also at least 10-fold cheaper to run than cells, and can be run in high-throughput as reactions are the equivalent of a reagent and used in a 384-well plate. Typical yields of prokaryotic systems are 750 μg/mL of GFP (30 μM). Cell-free systems can multiple organisms can be implemented and expression conducted at scales from 10 μl up to 10 mL.

Directions on how to make the extract component of cell-free systems, particularly lysates from E. coli, can be found in (Sun et al. 2013), which is hereby incorporated by reference herein in its entirety; other methods for producing a lysate are Known to one or ordinary skin in the art. While this procedure is adapted for E. coli cell-free systems, it can be used to produce other cell-free systems from other organisms and hosts (prokaryotic, eukaryotic, archaea, fungal, etc.) Examples, without limitation, of the production of other cell-free systems include Streptomyces spp. (Thompson et al. 1984), Bacillus spp. (Kelwick et al. 2016), and Tobacco BY2 (Buntru et al. 2014), which are hereby incorporated by reference herein in their entireties. Exemplary processes for producing lysates involve growing a host in a rich media to mid-log phase, followed by washes, lysis by French Press and/or Bead Beating Homogenization and/or equivalent method, and clarification. A lysate that has been processed as such can be referred to as a “lysate”, or a “treated cell lysate”, and is a non-limiting example of an “extract”. In an embodiment, cells may be grown under anaerobic conditions. In an aspect, an extract may be prepared under anaerobic conditions.

One or more additives may be supplied along-side an extract to maintain gene expression. Contemplated additives include those tailored to replicate the in vivo expression and/or the metabolic environment of the lysate source organism, e.g., redox buffering agents, phosphate potential buffering agents, customized energy regeneration systems, native ribosomes, chaperones, species-specific tRNAs, pH buffering, metals (such as Magnesium and Potassium), osmoregulatory agents, gas concentrations; [O2], [CO2], [N2], sugars, maltose, starch, maltodextrin, glucose, glucose-6-phosphate, fructose-1,6-biphosphate, 3-phosphoglycerate, phosphoenolpyruvate, pyruvate kinase, pyruvate dehydrogenase, pyruvate, acetyl phosphate, acetate kinase, creatine kinase, creatine phosphate, glutamate, amino acids, ATP, GTP, CTP, UTP, ADP, GDP, CDP, UDP, AMP, GMP, CMP, UMP, folinic acid, spermidine, putrescine, betaine, DTT, TCEP, b-mercaptoethanol, TPP, FAD, FADH, NAD, NADH, NADP, NADPH, oxalic acid, CoA, glutamate-salts, acetate-salts, cAMP, native polymerases, synthetic polymerases, phage polymerases, temperature regulation conditions. A review of optional additives can be found in (Chiao et al. 2016), which is hereby incorporated by reference herein in its entirety. Optional additives may also include components that assist transcription and translation, such as phage polymerases, T7 RNA polymerase (RNAP), SP6 phage polymerase, cofactors, elongation factors, nanodiscs, vesicles, and antifoaming agents. Optional additives may also include additives to protect DNA, such as, without limitation, gamS, Ku, junk DNA, DNA mimicry proteins. chi site-DNA, or other DNA protective agents.

In some embodiments, the reaction may include more than 0.1% (w/v) of crowding agent. Macromolecular crowding refers to the effects of adding macromolecules to a solution, as compared to a solution containing no macromolecules. Such macromolecules are termed crowding agents. A contemplated crowding agent may be from a single source, or may be a mix of different sources. The crowding agent may be from varied sizes. In some embodiments, the crowding agents include polyethylene glycol and its derivatives, polyethylene oxide or polyoxyethylene.

An energy recycling and/or regeneration system drives synthesis of mRNA and proteins by providing ATP to a system and by maintaining system homoeostasis by recycling ADP to ATP, by maintaining pH, and generally supporting a system for transcription and translation. A review of energy recycling systems can be found in (Chiao et al. 2016), which is hereby incorporated by reference herein in its entirety. Examples, without limitation, of energy recycling and/or systems that can be used include Glycerate 3-phosphate (3-PGA) (Sun et al. 2013), creatinine phosphate/creatinine kinase (CP/CK) (Kigawa et al. 1999), PANOx (Kim & Swartz 2001), and glutamate (Jewett & Swartz 2004). Other recycling and/or systems include those that can regenerate redox potential ([NAD(P)H]/[NAD(P+)]). An example of redox recycling is described in (Opgenorth et al. 2014). Recycling and/or systems can utilize innate central metabolism pathways from the host (for example, glycolysis, oxidative phosphorylation), externally supplied metabolic pathways, or both.

The in vitro transcription and translation system includes one or more nucleic acids. In an embodiment, the nucleic acid may include DNA, RNA, or combinations thereof. In some embodiments, a DNA may be supplied that that can produce a protein by utilizing transcription and translation machinery in the extract and/or additions to the extract. This DNA may have regulatory regions, such as under the OR2-OR1-Pr promoter (Sun et al. 2013), the T7 promoter or T7-lacO promoter, along with a RBS region, such as the UTR1 from lambda phage. The DNA may be linear or plasmid. In some embodiments, gene sequences may be engineered for cell-free expression in TXTL systems derived from the lysate source organism, such as: 5′ rare codons for improved TXTL coupling, 5′ AT/GC content for improved TXTL coupling, UTR, RBS, termination sequences, 5′ fusions for improved TXTL coupling, gene fusions for improved TXTL coupling, fusions for protein stability, sequence deletions to promote solubility of membrane proteins, and protein tags.

In other embodiments, a mRNA may be supplied that utilizes translational components in the lysate and/or additions to the lysate to produce a protein. The mRNA may be from a purified natural source, or from a synthetically generated source, or can be generated in vitro, e.g., from an in-vitro transcription kit such as HiScribe™, MAXIscript™, MEGAscript™, mMESSAGE MACHINE™, MEGAshortscript™.

In some embodiments, non-canonical amino acids may be utilized in the composition. Non-canonical amino acids may be found naturally in the cellular-produced product, or may be artificially added to the product to produce desirable properties, such as tagging, visualization, resistance to degradation, or targeting. While implementation of non-canonical amino acids is difficult in cells, in cell-free systems implementation rates are higher due to the ability to saturate with the non-canonical amino acid. Examples, without limitation, of non-canonical amino acids, including ornithine, norleucine, homoarginine, tryptophan analogs, biphenylalanine, hydrolysine, pyrrolysine, or as described in (Blaskovich 2016) which is hereby incorporated by reference herein in its entirety.

In some embodiments, the input nucleic acids are derived from extremophiles or anaerobes. The composition can produce the desired product using these environmental sequences by emulating the activity of the host cell (eg. in producing an anaerobic environment), thereby acting as an “artificial cell” or an alternate heterologous expression platform.

### Oxygen, Oxygen Radical, or Hydrogen Peroxide Scavengers

To maintain an anaerobic environment in a cell-free system, O₂, O⁻, or H₂O₂ scavengers can remove oxygen, oxygen radicals, and/or hydrogen peroxide from the cell-free system by either bioconversion or from binding and/or sequestration. This allows for the cell-free system to behave anaerobically, even if the host organism is not anaerobic or the physical conditions are not anoxic. In an embodiment, the composition for anaerobic in vitro transcription and translation may include one or more O₂, O⁻, or H₂O₂ scavengers. In an aspect, the one or more O₂, O⁻, or H₂O₂ scavengers include one or more binders of O₂, O⁻, or H₂O₂, one or more biochemical converters that biochemically convert O₂, O⁻, or H₂O₂ into another molecule, or combinations thereof.

### Enzyme, Protein, or Protein-Like Mimetic Scavengers

In some embodiments, the one or more scavengers may include an enzyme, protein, or protein-like mimetic that binds and/or sequesters O₂, O⁻, or H₂O₂. In an embodiment, the binding and/or sequestration of O₂, O⁻, or H₂O₂ may be reversible or irreversible. Examples, without limitation, of contemplated enzymes and proteins that are O₂, O⁻, or H₂O₂ scavengers include catalase, superoxide dismutase, peroxidase, hemoglobin, myoglobin, porphyrin, oxidase, oxygenase, rubisco, and mimetics thereof. These enzymes and proteins may be naturally occurring (isolated from the environment), engineered variants, or synthetically generated to mimic the naturally-occurring variant. In some embodiments, the enzyme, protein, or protein-like mimetic is a homolog or variant of a known entity that binds oxygen.

In some embodiments, the enzyme, protein, or protein-like mimetic may include one or more a transition metals. In an embodiment, the transition metal may be in ionic form; the transition metal may have a charge of 2+ or 3+. In an aspect. the transition metal may be Iron; in a further aspect, the transition metal may be Aluminum, Copper, Cobalt, Tin, Lead, Vanadium, Chromium, and other transition metals without limitation. The transition metal may be within a coordination complex with other molecules such as porphyrin. The enzyme, protein, or protein-like mimetic may be naturally occurring. The enzyme, protein, or protein-like mimetic may also be engineered to contain a non-native transition metal.

In some embodiments, the enzyme, protein, or protein-like mimetic may include one or more heme groups. Within a heme, an iron ion is coordinated to a porphyrin acting as a tetradentate ligand, and to one or two axial ligands. These enzymes, proteins, or protein-like mimetics may also be known as hemoproteins. Multiple types of heme groups exist, such as heme A, heme B, heme C, heme O, Heme I, heme m, heme D, heme S.

### Conversion of Oxygen, Oxygen Radical, or Hydrogen Peroxide

In some embodiments, the one or more scavengers may biochemically convert O₂, O⁻, or H₂O₂ into another molecule. In an embodiment, the conversion may be irreversible. An exemplary scavenger, cytochrome oxidase, transfers electrons to molecular oxygen to generate two molecules of water. Another exemplary scavenger, glucose oxidase, oxidizes glucose to form D-glucono-1,5-lactone and hydrogen peroxide. Another exemplary scavenger, monooxygenase, reduces two molecules of oxygen to form one hydroxyl group and one molecule of water. Another exemplary scavenger, catalase, catalyzes the decomposition of two molecules or hydrogen peroxide to form two molecules of water and one molecule of oxygen.

In an embodiment, the composition for anaerobic in vitro transcription and translation may also include one or more additives that facilitate the function of the scavenger. The scavenger additive may be an additional cofactor, coenzyme, energy source, or the like. The cofactor may affect enzyme catalysis, enzyme structure, or both. The scavenger additive may assist the activity of an enzyme. In an embodiment, the scavenger additive may be selected from the group consisting of: ATP/ADP, NAD/NADH, NADP/NADPH, FAD/FADH, and the like. In an embodiment, the additive may be a substrate for a scavenger enzyme. An exemplary, non-limiting, substrate additive is glucose.

In some embodiments, the scavenger system itself, starting substrates, intermediates, or products may be toxic to the cell-free system. In some embodiments, the toxic product may be more specifically, hydrogen peroxide. In the case of hydrogen peroxide, catalase can be used to irreversibly convert the product to water and oxygen. In an embodiment, the scavenger may include peroxidase. Peroxidases, such as horseradish peroxidase, can also be used to convert substrates such as AMPLEX® red to resorufin using excess hydrogen peroxide. In other embodiments, the toxic starting substrate, intermediate, or product may cause a shift in pH from a permissible pH to a non-permissible pH. An example of a starting substrate is glucose, which oxidizes to gluconic acid. To counteract the effects of glucose oxidation on pH, additional buffering capacity can be built into the extract using commonly used biocompatible buffers (e.g., HEPES, Bis-Tris, MOPS).

In some embodiments, the components of the scavenger system may include glucose, glucose oxidase, and catalase, as depicted in FIG. 3. Within this exemplary non-limiting buffering system, one unit of glucose and one oxygen is converted to a relatively inert substance, one unit of D-glucono-1,5-lactone and one unit of hydrogen peroxide. The hydrogen peroxide is toxic; two units of hydrogen peroxide are then removed from the system by catalase to generate two units of water and one unit of oxygen. For every two units of oxygen consumed, one is produced. As the reaction is irreversible, the resulting system accumulates D-glucono-1,5-lactone and becomes anaerobic. To counteract pH changes from accumulation of carboxylic acids, additional buffering capability can be added to the cell-free system. In other embodiments, glucose oxidase is substituted for pyranose oxidase, producing dehydro-D-glucose rather than D-glucono-1,5-lactone.

In an embodiment, the scavenger system may include glucose at a concentration or 1 nM to 5 M; 50 nM to 500 mM; or 50 μM to 200 mM. In an embodiment, the scavenger system may include glucose oxidase at a concentration of 0.1 μM to 1 M; 0.1 nM to 1 mM; or 1 nM to 500 μM. In an embodiment, the scavenger system may include catalase at a concentration of 0.1 pM to 1 M; 0.1 nM to 1 mM; or 1 nM to 500 μM. In an embodiment, the scavenger system may include pyranose oxidase at a concentration of 0.1 μM to 1 M; 0.1 nM to 1 mM; or 1 nM to 500 μM.

In other exemplary non-limiting embodiments, the components of the scavenger system include protocatechuate 3,4-dioxygenase and protocatechuic acid. One unit of protocatechuic acid is reacted with oxygen to produce 3-carboxy-cis,cis-muconate. The production of carbocyclic acids that affect the cell-free system can be controlled by additional buffering capacity.

In an embodiment, the scavenger system may include protocatechuate 3,4-dioxygenase at a concentration of 0.1 μM to 1 M; 0.1 nM to 1 mM; or 1 nM to 500 μM. In an embodiment, the scavenger system may include protocatechuic acid at a concentration of 1 nM to 5 M; 50 nM to 500 mM; or 50 μM to 200 mM.

Binding of Oxygen, Oxygen radical, or Hydrogen Peroxide

In some exemplary non-limiting embodiments, the components of the scavenger system bind or chelate oxygen, oxygen radicals, and/or hydrogen peroxide, effectively sequestering it from interacting with other components in solution. In an embodiment, the binding may be reversible. An example of a molecule that naturally binds oxygen is hemoglobin, which is used for oxygen transport in vertebrates and invertebrates. Within hemoglobin are heme groups containing iron held in a porphyrin heterocyclic ring, that are able to reversibly bind oxygen in a coordinate covalent bond. In an embodiment, in a cell-free system, hemoglobin may be added to the cell-free system in excess to reduce oxygen concentration in the cell-free system. In some embodiments, exemplary scavengers may be oxygen-carrier proteins, and are known as hemoglobin, hemerythrin, or hemocyanins. In other embodiments, the scavengers may be synthetic or engineered. In some embodiments, the scavengers may include heme groups.

In some embodiments, the level of oxygen binding can be titrated by additional additives. Using hemoglobin as an example, the amount of oxygen bound to hemoglobin can be described by the oxygen hemoglobin dissociation curve, and different variables (eg. pH, temperature, 2,3-DPG) can shift the affinity of hemoglobin for oxygen. Therefore, decreased temperature, higher pH, or decreased 2,3-DPG concentrations can encourage oxygen binding.

### Using Oxygen, Oxygen Radical, or Hydrogen Peroxide Scavengers in Other In Vitro Environments

A further embodiment relates to cell-free oxygen-deprived enzymatic systems. The cell-free oxygen-deprived enzymatic systems include one or more scavengers described herein.

As understood by one of ordinary skill in the art, the scavengers described herein are useful not only for cell-free transcription and translation reactions, but also for any in vitro enzymatic reaction. The scavengers provide a biochemical method to reduce oxygen, oxygen radical, and hydrogen peroxide concentrations in any aqueous solutions. Therefore, as long as the substrates, inputs, outputs, or intermediates are compatible with the reaction in the aqueous solution, the scavengers can provide benefit.

In some embodiments, the cell-free oxygen-deprived enzymatic systems include purified enzymes where enzymes are combined together to produce a product, either from an exogenously supplied input or from the enzymes themselves. These in vitro compositions do not have to conduct transcription and/or translation to produce products, with an exemplary example, without limitation, described in (Opgenorth et al. 2014), which is hereby incorporated by reference herein in its entirety. Examples in the literature, without limitation, include the conversion of glucose and/or other sugars to bioplastics, terpenoid-like molecules (isoprene, limonene), to hydrogen, to tagatose, and to alluose. Purified enzymes, added together with an energy and/or redox potential regeneration system, can convert inputs to outputs at high concentrations (mg/mL). For many of these reactions, the enzymes involved or the substrates may require anaerobic conditions to function properly, or the products themselves are derived from anaerobic organisms and require similar anaerobic conditions to operate. However, physical methods of achieving anaerobic conditions are uneconomical or impractical. In lieu, the scavengers proposed herein can be used instead to provide a biochemical alternative.

In some embodiments, the cell-free oxygen-deprived enzymatic systems include combinations of lysates derived from one or more natural or engineered organisms that are mixed together to produce a product, either from an exogenously supplied input or from the components of the lysates themselves. Within each lysate, a necessary enzyme or pathway may be overproduced by genetic engineering methods. An example, without limitation, is described as “cell-free metabolic engineering” in (Opgenorth et al. 2014; Karim & Jewett 2016), which are hereby incorporated by reference herein in their entireties. Further, in some embodiments, the in vitro compositions are combinations of both lysates and purified enzymes.

### EXAMPLES

### Example 1: Expression of Genes Derived from Anaerobic Organism or Environment in an Archetypal Oxygen-Deprived In Vitro System

In vivo, TXTL in anaerobes operates in a cytoplasm that the cell maintains at low redox potential, i.e, at low oxidizing conditions. Anaerobes have O2-sensitive enzyme activities that have evolved to maintain operation of non-respiratory metabolism, ex. hydrogenases, radical-assisted enzyme catalysis, diazotrophy, etc.

An exemplary in vitro TXTL system composition:
Treated lysate prepared from anaerobic bacterial host, e.g., Clostridium sp. (Clostridium cultured under strict anaerobic conditions, lysate prepared using standard technique in enclosed hood under N2 atmosphere, lysate addressed in 25 mL aliquots into 384-well microtiter plate under N2 atmosphere) OR lysate prepared from standard bacterial host under aerobic or anaerobic conditions.
Standard additions for transcription and translation.
Additions to maintain reducing, low O₂ environment for expression: NAD(P)H/NAD(P)+ poising system to maintain low redox potential lysate at ratio>100:1, NAD(P)H regenerating transhydrogenase for redox recycling, O₂ scavengers: hemoglobin, oxidase or oxygenase enzymes, reactive oxygen scavengers: catalase, peroxidase, superoxide dismutase, Substrate for ATP regeneration specific for the host organism metabolic pathway.

### Example 2. Oxygen-Deprived Conditions can be Simulated in a Cell-Free System

To stimulate anaerobic conditions, various additives that contribute to enzyme maturation and to oxygen scavenging were added to the reaction. One sample system is a oxygen scavenging system composed of glucose, glucose oxidase, and catalase shown in FIG. 3, where for every two molar of oxygen consumed 1 molar of oxygen is produced, and where catalase is able to recycle toxic intermediate hydrogen peroxide. The production or D-glucono-1,5-lactone is relatively inert to cell-free systems. As each reaction is irreversible, the reaction shifts towards oxygen depletion. The addition and subsequent flux of glucose in this sample system can cause pH changes in the resulting reaction, making it important to provide buffering ability in the cell-free system.

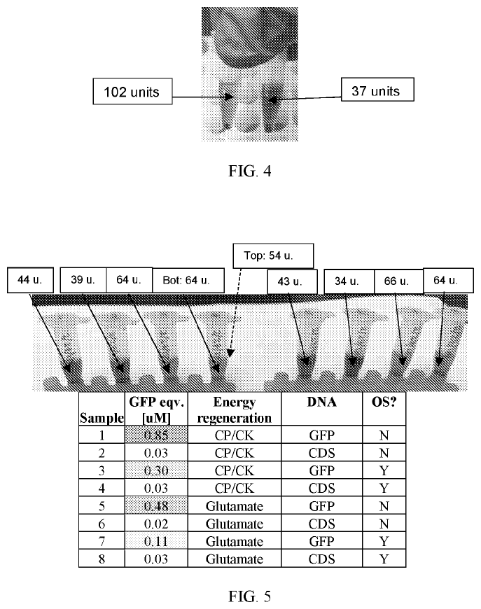

In one reaction, the following oxygen scavenging stocks were used: a 100× stock solution of glucose oxidase to 10 μM, 100× stock solution of catalase to 150 μM, and 10× stock solution of glucose to 500 mM. Two 200 μL cell-free expressions were set up to test the effect of oxygen scavenging on the free iron oxidation condition. In FIG. 4, the results of adding the oxygen scavenging stocks is compared in two different reactions: one with oxygen scavenging at 1×working concentration (0.1 μM glucose oxidase, 1.5 μM catalase, 50 mM glucose), and one without oxygen scavenging. Each reaction was otherwise similar, utilizing E. coli TXTL eAC28 and buffers produced by methods described in (Sun et al. 2013), with the following modifications: pH buffering using 90 mM Bis-Tris (vs. HEPES) and the addition of 2 mM pyridoxal phosphate, 2 mM L-Cysteine, 1 mM Fe²⁺, 1 mM Dithiothreitol, and 1 mM Sodium sulfide. After an incubation period of hours at 29° C., a difference in color, showing a pale color in the oxygen scavenged sample (left) vs. a dark color in the in the non-oxygen scavenged sample (right), indicated the decrease of available oxygen based on the oxidation state of the free iron in the reaction. To determine a quantitative metric, points indicated by the arrow were measured for intensity in arbitrary units using ImageJ's “measure” feature, where higher numbers indicate brighter areas. The oxygen scavenged sample measured 102 units vs. the non-oxygen scavenged sample which measured 37 units. This showed that biochemical oxygen scavenging additives can remove oxygen from cell-free systems. Unless otherwise noted, all chemicals were obtained from Sigma-Aldrich. ImageJ software is available at imagej.nih.gov/ij/.

### Example 3. Cell-Free Systems with Oxygen-Deprived Conditions Create Functional Protein

While cell-free systems were made oxygen-deprived using oxygen scavenging solutions, the system must also be capable of producing functional protein through the activity of transcription and translation. This may require tuning properties of the cell-free system. For example, different energy regeneration solutions can be used in cell-free systems that complement the anaerobic reaction condition. Energy regeneration utilizing a glutamate system uses oxidative phosphorylation, which is oxygen dependent. However, energy regeneration utilizing creatinine phosphate/creatinine kinase (CP/CK) uses substrate-level phosphorylation, which is oxygen independent.

In one example, eight different E. coli cell-free reactions expressing either GFP or another coding sequence were setup in FIG. 5. All reactions were 200 μL and share: 30% eAC28 E. coli lysate, produced by methods described in (Sun et al. 2013), 2 mM pyridoxal phosphate, 2 mM L-Cysteine, 1 mM Fe2+, and 1 mM Dithiothreitol. Reactions with oxygen scavenging (OS) also contained 0.1 μM glucose oxidase, 1.5 μM catalase, 50 mM glucose. Reactions with GFP DNA contained 8 nM of sigma70-GFP plasmid (Addgene #40019), while reactions with CDS DNA contained 8 nM of sigma70-non-fluorescent proteins total DNA as a control. Reactions with the CP/CK energy system utilized 35% energy solution buffer as described in (Sun et al. 2013), but with 30 mM creatinine phosphate and 0.2 mg/ml creatinine kinase in lieu of 3-PGA, and 90 mM of Bis-Tris buffering in lieu of HEPES. Reactions containing the glutamate energy system utilized 35% energy solution buffer as described in (Sun et al. 2013), but with 90 mM of Bis-Tris buffering in lieu of HEPES. Each reaction was kept in 0.65 mL centrifuge tubes, stored overnight at 29° C., and imaged the following day (FIG. 5, top). Of each sample, 10 μL was then removed and put into a Nunc 384-well plate, and imaged on a Biotek Synergy 2 using 485/ex 528/em filters. The resulting numbers were converted to an absolute standard of purified GFP. This experiment demonstrated that there is still a clear color change from the change in oxidation state of free iron when oxygen scavenging was added. To determine a quantitative metric, points indicated by the arrow were measured for intensity in arbitrary units using ImageJ's “measure” feature, where higher numbers indicate brighter areas. Samples subject to oxygen scrubbing measured 44, 39, 43, and 34 units, while those without oxygen scrubbing measured 64, 64, 66, and 64 units. Note that at the dotted black arrow, the interface of the oxygen-deprived cell-free system with the airspace, the color of the free iron was darker (measuring 54 units vs. 64 units in the oxygen scrubbed area), indicating not fully anaerobic conditions at the interface. The experiment also demonstrated that while oxygen scavenging can decrease the efficiency of protein production (due to toxicity, lower energy regeneration, or only trace oxygen to mature a fluorophore), functional protein (as measured by GFP fluorescence) was still produced, especially with energy regeneration systems that do not require oxygen. Therefore, oxygen scavenging systems still allow for functional protein production through active transcription and translation. Unless otherwise noted, all chemicals were obtained from Sigma-Aldrich.

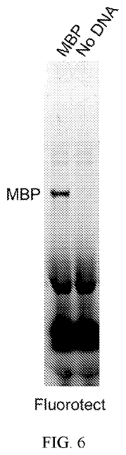

In another experiment, protein production was measured by directly detecting production using non-oxygen-dependent methods. Specifically, we measured the expression of sigma70-MBP (SEQ ID NO:1) using FluoroTect™, a lysine-charged tRNA labeled with BODIPY-FL, within oxygen scavenging conditions. Two 65 μL reactions in 0.2 mL PCR tubes were conducted. Each reaction contained 30% eEC4 E. coli lysate and 35% bAC10 buffer, produced by methods described in (Sun et al. 2013), 1% FluoroTect™, 20 μg/ml gamS, 2 mM pyridoxal phosphate, 2 mM L-Cysteine, 1 mM Fe2+, and 1 mM Dithiothreitol. Reactions also contained oxygen scavenging (OS) solutions 0.1 μM glucose oxidase, 1.5 μM catalase, 50 mM glucose. One reaction with MBP DNA contained 13.5 nM of sigma70-MBP linear DNA, while the other reaction contained no DNA as a control. The reaction changed color within 15 minutes from oxygen scrubbing and the change in oxidation state in Iron. After an overnight incubation at 29° C., 2 μL sample was analyzed on a 4-12% MES SDS-PAGE gel, and imaged for FluoroTect™ detection. In FIG. 6, MBP was clearly detected in the oxygen scavenging system, while no DNA control lacked MBP. Therefore, oxygen scavenging systems still allowed for functional protein production through active transcription and translation, also when measured on a non-oxygen dependent output. Unless otherwise noted, all chemicals were obtained from Sigma-Aldrich.

### EQUIVALENTS

The present disclosure provides among other things cell-free systems and use thereof. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications, patents and sequence database entries mentioned herein are hereby incorporated by reference in their entirety as if each individual publication or patent was specifically and individually indicated to be incorporated by reference.

### REFERENCES

Blaskovich, M. A. T., 2016. Unusual Amino Acids in Medicinal Chemistry. Journal of medicinal chemistry, 59(24), pp. 10807-10836.
Buntru, M. et al., 2014. Tobacco BY-2 cell-free lysate: an alternative and highly-productive plant-based in vitro translation system. BMC biotechnology, 14(1), p. 37.
Chiao, A. C., Murray, R. M. & Sun, Z. Z., 2016. Development of prokaryotic cell-free systems for synthetic biology. bioRxiv, p. 048710.
Jewett, M. C. & Swartz, J. R., 2004. Mimicking the Escherichia coli cytoplasmic environment activates long-lived and efficient cell-free protein synthesis. Biotechnol Bioeng, 86(1), pp. 19-26.
Karim, A. S. & Jewett, M. C., 2016. A cell-free framework for rapid biosynthetic pathway prototyping and enzyme discovery. Metab Eng, 36, pp. 116-126.
Kelwick, R. et al., 2016. Development of a Bacillus subtilis cell-free transcription-translation system for prototyping regulatory elements. Metab Eng, 38, pp. 370-381.
Kigawa, T. et al., 1999. Cell-free production and stable-isotope labeling of milligram quantities of proteins—Kigawa—1999—FEBS Letters—Wiley Online Library. FEBS . . . .
Kim, D.-M. & Swartz, J. R., 2001. Regeneration of adenosine triphosphate from glycolytic intermediates for cell-free protein synthesis. Biotechnol Bioeng, 74(4), pp. 309-316.
Opgenorth, P. H., Korman, T. P. & Bowie, J. U., 2014. A synthetic biochemistry molecular purge valve module that maintains redox balance. Nature Communications, 5, p. 998.
Sun, Z. Z. et al., 2014. Linear DNA for Rapid Prototyping of Synthetic Biological Circuits in an Escherichia coli Based TX-TL Cell-Free System. ACS Synth Biol, 3(6), pp. 387-397.
Sun, Z. Z. et al., 2013. Protocols for Implementing an Escherichia Coli Based TX-TL Cell-Free Expression System for Synthetic Biology. Journal of Visualized Experiments, e50762(79), pp. e50762-e50762.
Thompson, J., Rae, S. & Cundliffe, E., 1984. Coupled transcription—translation in extracts of Streptomyces lividans. Molecular and General Genetics MGG, 195(1-2), pp. 39-43.

SEQ ID NO: 1
AAAACCGAATTTTGCTGGGTGGGCTAACGATATCCGCCTGATGCGTGAAC
GTGACGGACGTAACGAAGACAACCACGCATTGCTGTTCTGAGCTAACACC
GTGCGTGTTGACAATTTTACCTCTGGCGGTGATAATGGTTGCAGCAAGCA
ATAATTTTGTTTAACTTTAAGAAGGAGATATACAATGAAAATCGAAGAAG
GTAAACTGGTAATCTGGATTAACGGCGATAAAGGCTATAACGGCCTCGCT
GAAGTCGGTAAGAAATTCGAGAAAGATACCGGAATTAAAGTCACCGTTGA
GCATCCGGATAAACTGGAAGAGAAATTCCCACAGGTTGCGGCAACTGGCG
ATGGCCCTGACATTATCTTCTGGGCACACGACCGCTTTGGTGGCTACGCT
CAATCTGGCCTGTTGGCTGAAATCACCCCGGACAAAGCGTTCCAGGACAA
GCTGTATCCGTTTACCTGGGATGCCGTACGTTACAACGGCAAGCTGATTG
CTTACCCGATCGCTGTTGAAGCGTTATCGCTGATTTATAACAAAGATCTG
CTGCCGAACCCGCCAAAAACCTGGGAAGAGATCCCGGCGCTGGATAAAGA
ACTGAAAGCGAAAGGTAAGAGCGCGCTGATGTTCAACCTGCAAGAACCGT
ACTTCACCTGGCCGCTGATTGCTGCTGACGGGGGTTATGCGTTCAAGTAT
GAAAACGGCAAGTACGACATTAAAGACGTGGGCGTGGATAACGCTGGCGC
GAAAGCGGGTCTGACCTTCCTGGTTGACCTGATTAAAAACAAACACATGA
ATGCAGACACCGATTACTCCATCGCAGAAGCTGCCTTTAATAAAGGCGAA
ACAGCGATGACCATCAACGGCCCGTGGGCATGGTCCAACATCGACACCAG
CAAAGTGAATTATGGTGTAACGGTACTGCCGACCTTCAAGGGTCAACCAT
CCAAACCGTTCGTTGGCGTGCTGAGCGCAGGTATTAACGCCGCCAGTCCG
AACAAAGAGCTGGCAAAAGAGTTCCTCGAAAACTATCTGCTGACTGATGA
AGGTCTGGAAGCGGTTAATAAAGACAAACCGCTGGGTGCCGTAGCGCTGA
AGTCTTACGAGGAAGAGTTGGCGAAAGATCCACGTATTGCCGCCACCATG
GAAAACGCCCAGAAAGGTGAAATCATGCCGAACATCCCGCAGATGTCCGC
TTTCTGGTATGCCGTGCGTACTGCGGTGATCAACGCCGCCAGCGGTCGTC
AGACTGTCGATGAAGCCCTGAAAGACGCGCAGACTCGTATCACCAAGGGT
GGATCTGGATGTTGTCCTGGCTGTTGCTGATGAATAACTGAATAGGGGAT
CCCGACTGGCGAGAGCCAGGTAACGAATGGATCCCCGAGCTCGAGCAAAG
CCCGCCGAAAGGCGGGCTTTTCTGTCCTTGAGAGTCGGGCATTGTCTTCG
CTCCTTCCGGTGGGCGCGGGGCATGACTATCGTCGCCGCACTTATGACTG
TGTTCTTTATCAT

1. A cell-free composition, comprising:
an extract derived from one or more organisms;
one or more proteins of interest, wherein the one or more proteins are expressed from one or more nucleic acids exogenous to the extract and/or by the one or more organisms, wherein preferably the one or more proteins react with a substrate to produce a product; and
one or more O₂, O⁻, or H₂O₂ scavengers.

2. The composition of claim 1, wherein the composition further comprises the one or more nucleic acids and is for in vitro transcription and translation of the one or more proteins from the one or more nucleic acids, wherein preferably the one or more nucleic acids comprises a gene or a fragment thereof derived from an extremophile or anaerobe.

3. The composition of claim 1, wherein the one or more organisms are genetically engineered to express the one or more proteins.

4. A composition for in vitro transcription and translation, comprising:
a first set of cofactors, enzymes, and other reagents necessary for transcription and/or translation;
a second set of cofactors, enzymes, and other reagents necessary for energy recycling;
one or more nucleic acids encoding one or more proteins of interest; and
one or more O₂, O⁻, or H₂O₂ scavengers.

5. The composition of claim 4, wherein the first set of proteins, enzymes, and other reagents are wholly or partially provided by an extract derived from one or more organisms and/or from a fully or partially purified source.

6. The composition of claim 4, wherein the second set of proteins, enzymes, and other reagents are wholly or partially provided by an extract derived from one or more organisms and/or from a fully or partially purified source.

7. The composition of claim 1, wherein the composition is oxygen-deprived or anaerobic.

8. The composition of claim 1, wherein the one or more organisms are selected from bacteria, archaea, plants, and animals.

9. The composition of claim 8, wherein the one or more organisms are selected from extremophiles and Clostridium.

10. The composition of claim 1, wherein the one or more O₂, O⁻, or H₂O₂ scavengers binds O₂, O⁻, or H₂O₂ and/or biochemically converts (e.g., reduces or oxidizes) O₂, O⁻, or H₂O₂ into another molecule.

11. The composition of claim 10, wherein the one or more O₂, O⁻, or H₂O₂ scavengers comprises a heme group, and is preferably selected from the group consisting of: catalase, superoxide dismutase, peroxidase, hemoglobin, myoglobin, porphyrin, oxidase, oxygenase, rubisco, and homologs or variants thereof.

12. The composition of claim 11, wherein the one or more O₂, O⁻, or H₂O₂ scavengers further comprises a transition metal bound by the heme group.

13. The composition of claim 11, wherein the one or more O₂, O⁻, or H₂O₂ scavengers is selected from the group consisting of: glucose oxidase, pyranose oxidase, protocatechuate 3,4-dioxygenase and catalase.

14. The composition of claim 1, further comprising an exogenous energy recycling system.

15. The composition of claim 14, wherein the exogenous energy recycling system comprises one or more components selected from the group consisting of:
components for providing redox potential, components for providing phosphate potential, and combinations thereof.

16. The composition of claim 1, further comprising one or more additives, preferably selected from one or more cofactors, enzymes, and other reagents necessary for transcription and/or translation.

17. The composition of claim 1, wherein the extract comprises one or more of: a whole cell extract, a nuclear extract, a cytoplasmic extract, and mixtures thereof, wherein preferably the extract comprises a whole cell lysate.

18. A method for protein synthesis, comprising:
(a) providing the composition of claim 1; and
(b) isolating the one or more proteins from the composition.

19. The method of claim 18, further comprising expressing the one or more proteins from the one or more nucleic acids in vitro in the composition.

20. A method for in vitro preparation of a product, comprising:
(a) providing the composition of claim 1;
(b) allowing the one or more proteins to react with the substrate to produce the product; and
(c) optionally, isolating the product from the composition.
